(19) United States
(12) Patent Application Publication
(10) Pub. No.: US 2004/0143093 A1
(43) Pub. Date: Jul. 22, 2004

(54) Method for inducing a conformational transition in proteins, such as pathogenic/infectious proteins, and their use

(76) Inventors: Ralph Zahn, Zurich (CH); Thorsten Luhrs, San Diego, CA (US)

Correspondence Address: MOETTELI & ASSOCIES SARL, CASE POSTALE 486, AVE DE FRONTENEX 6, GEVEVA 12, CH-1211, CH

(21) Appl. No.: 10/612,356

(22) Filed: Jul. 2, 2003

Related U.S. Application Data
(60) Provisional application No. 60/395,203, filed on Jul. 11, 2002.

(51) Int. Cl. C07K 14/47

(52) U.S. Cl. 530/350; 435/69.1; 435/320.1; 435/325

(57) ABSTRACT

The invention relates to an in vitro method for inducing a conformational transition in proteins, whereas said conformational transition results in an increased content of β-sheet secondary structure, the method comprising the steps of:

a) providing a conversion buffer; b) adding a solution of lamellar lipid structures that comprise negatively charged lipids to the conversion buffer; c) adding protein molecules to the conversion buffer; d) forming a sample mixture from the conversion buffer, the added lipids and protein molecules; e) establishing a conversion temperature in the sample mixture; and f) exposing the sample mixture of step d) to the conversion temperature according to step e) for a time sufficient to form conformationally transitioned proteins. By this method water soluble complexes of lamellar lipidic structures and conformationally transitioned proteins are formed, the conformationally transitioned proteins being oligomeric β-sheet intermediate structures. Amyloidogenic aggregates may be produced of the water soluble complexes of lamellar lipidic structures and oligomeric β-sheet intermediate structures by actively destroying the lamellar lipid structures. Such proteins may be involved in neurodegenerative diseases like Transmissible Spongiform Encephalopathy (TSE), Alzheimers disease, Multiple Sclerosis and Parkinsons disease. The disclosure comprises the use of the proteins produced by the method, e.g., for exploiting the various aspects of the PrP^{C} to PrP^{Sc} conversion; for the development of new diagnostic TSE-tests and potential therapeutics or prophylactics against TSE such as Creutzfeldt-Jakob disease in human.

### RELATED APPLICATION DATA

This patent application claims priority of the U.S. provisional application No. 60/395,203 filed on Jul. 11, 2002, the entire disclosure of which is incorporated herein by reference.

### BACKGROUND OF THE INVENTION

Although the central paradigm of protein folding (Anfinsen, C. B. (1973) Principles That Govern Folding of Protein Chains. Science, 181, 223-230), that the unique three-dimensional structure of a protein is encoded in its amino acid sequence, is well established, its generality has been questioned due to the recently developed concept of “prions”. Biochemical characterization of infectious scrapie material causing central nervous system degeneration indicates that the necessary component for disease propagation is proteinaceous (Prusiner, S. B. (1982) Novel proteinaceous infectious particles cause scrapie. Science, 216, 136-144), as first outlined by (Griffith, J. S. (1967) Self-replication and scrapie. Nature, 215, 1043-1044) in general terms. Prion propagation further involves a conversion from a cellular prion protein, denoted PrP^{C}, into a toxic scrapie form, PrP^{Sc}, which is facilitated by PrP^{Sc} acting as a template for PrP^{C} to form new PrP^{Sc} molecules (Prusiner, S. B. (1987) Prions and neurodegenerative diseases. N Engl J Med, 317, 1571-1581). The “protein-only” hypothesis implies that the same polypeptide sequence, in the absence of any post translational modifications, can adopt two considerably different stable protein conformations. Thus, in the case of prions it is possible, although not proven, that they violate the central paradigm of protein folding. There is some indirect evidence that another factor might be involved in the conformational conversion process (Prusiner, S. B. (1998) Prions. Proc Natl Acad Sci USA, 95, 13363-13383), which includes a dramatic change from α-helical into β-sheet secondary structure. Although it has been proposed that a presumed “factor X” might act as a molecular chaperone, its chemical nature has not been identified yet (Zahn, R. (1999) Prion propagation and molecular chaperones. Q Rev Biophys, 32, 309-370). “Factor X”, thus, could be a protein, a lipid, another biological macromolecule, or a combination thereof.

Two general models have been proposed for the molecular mechanism by which PrP^{Sc} promotes the conversion of the cellular isoform (see FIG. 1). The “nucleated polymerization” or “seeding” model for PrP^{Sc} formation (Jarrett, J. T. and Lansbury, P. T., Jr. (1993) Seeding “one-dimensional crystallization” of amyloid: a pathogenic mechanism in Alzheimer's disease and scrapie? Cell, 73, 1055-1058) proposes that PrP^{C} and PrP^{Sc} are in a rapidly established equilibrium, and that the conformation of PrP^{Sc} is thermodynamically stable only when trapped within a crystal-like seed (see FIG. 1A). The proposed process is akin to other well-characterized nucleation-dependent protein polymerization processes, including microtubule assembly, flagellum assembly, and sickle-cell hemoglobin fibril formation, where the kinetic barrier is imposed by nucleus formation around single molecules. To explain exponential conversion rates, it must be assumed that the aggregates are continuously fragmented to present increasing surface for accretion, although the mechanism of fragmentation remains to be explained. The “template-assisted” or “heterodimer” model for PrP^{Sc} formation (Prusiner, S. B., Scott, M., Foster, D., Pan, K. M., Groth, D., Mirenda, C., Torchia, M., Yang, S. L., Serban, D., Carlson, G. A. and et al. (1990) Transgenetic studies implicate interactions between homologous PrP isoforms in scrapie prion replication. Cell, 63, 673-686) proposes that PrP^{C} is unfolded to some extent and refolded under the influence of a PrP^{Sc} molecule functioning as a template (see FIG. 1B). A high energy barrier is postulated to make this conversion improbable without catalysis by preexisting PrP^{Sc}. The conformational change is proposed to be kinetically controlled by the dissociation of a PrP^{C}-PrP^{Sc} heterodimer into two PrP^{Sc} molecules, and can be treated as an induced fit enzymatic reaction following autocatalytic Michaelis-Menten kinetics. Once conversion has been initiated it gives rise to an exponential conversion cascade as long as the PrP^{Sc} dimer dissociates rapidly into monomers. A disadvantage of the template-assisted model is that it does not explain why PrP^{Sc} after propagation should aggregate into protein fibrils. Manfred Eigen has presented a comparative kinetic analysis of the two proposed mechanisms of prion disease (Eigen, M. (1996) Prionics or the kinetic basis of prion diseases. Biophysical Chemistry, 63, A1-A18). He found that logically both models are possible, in principle, but that the conditions under which they work seem to be too narrow and unrealistic. The autocatalytic template-assisted model requires cooperativity in order to work, but it then becomes phenomenologically indistinguishable from the nucleation model which is also a form of (passive) autocatalysis. Though the two kind of mechanisms still may differ on the question which of the two monomeric protein conformations is the favored equilibrium state, they both require an aggregated state as the from that is eventually favored at equilibrium and that presumably resembles the pathogenic form of the prion protein. Eigen concluded that more experimental evidence is needed in order to judge which of the two models is the right one. In principle, neither of the models for prion propagation does rule out a possible assistance by “factor X”.

A mechanistic understanding of prion diseases requires a detailed knowledge of the three-dimensional structure of both the cellular form and the pathogenic form of the prion protein. Only if both protein structures have been deciphered one can understand how a conversion takes place. In vivo, the “healthy” prion protein is attached to the cell surface via a glycosyl phosphatitylinositol anchor and partitions to membrane domains that have been termed lipid rafts (Vey, M., Pilkuhn, S., Wille, H., Nixon, R., DeArmond, S. J., Smart, E. J., Anderson, R. G., Taraboulos, A. and Prusiner, S. B. (1996) Subcellular colocalization of the cellular and scrapie prion proteins in caveolae-like membranous domains. Proc Natl Acad Sci USA, 93, 14945-14949). Recent structural studies have focused on soluble recombinant prion proteins from various species using nuclear magnetic resonance (NMR) spectroscopy. These studies show that mammalian PrP^{C} consists of two distinct domains: a flexibly disordered N-terminal tail, which comprises residues 23-120, and a well structured C-terminal globular domain of residues 121-230 that is rich in α-helix secondary structure and contains a small anti-parallel β-sheet (Lopez Garcia, F., Zahn, R., Riek, R. and Wüthrich, K. (2000) NMR structure of the bovine prion protein. Proc Natl Acad Sci USA, 97, 8334-8339). Upon conversion of PrP^{C} into PrP^{Sc}, residues 90-120, which represent the most conserved sequence element in mammalian and non-mammalian prion proteins (Wopfner, F., Weidenhofer, G., Schneider, R., von Brunn, A., Gilch, S., Schwarz, T. F., Werner, T. and Schätzl, H. M. (1999) Analysis of 27 mammalian and 9 avian prion proteins reveals high conservation of flexible regions of the prion protein. J Mol Biol, 289, 1163-1178), become resistant to treatment with proteinase K (Prusiner, S. B., Groth, D. F., Bolton, D. C., Kent, S. B. and Hood, L. E. (1984) Purification and structural studies of a major scrapie prion protein. Cell, 38, 127-134), implying that this polypeptide segment becomes structured. There is further evidence that the conformational transition of PrP^{C} is accompanied by a substantial increase of the α-sheet secondary structure (Pan, K. M., Baldwin, M., Nguyen, J., Gasset, M., Serban, A., Groth, D., Mehlhorn, I., Huang, Z., Fletterick, R. J., Cohen, F. E. and et al. (1993) Conversion of alpha-helices into beta-sheets features in the formation of the scrapie prion proteins. Proc Natl Acad Sci USA, 90, 10962-10966).

### PROBLEMS OBSERVED IN PRIOR ART

Clearly defining the conformational properties of different forms of PrP is crucial to defining the transition and disease mechanisms. In the case of prions this proves challenging because the most powerful methods for determining protein conformation rely on soluble homogenous samples precluding the investigation of aggregates. So far, pathogenic prion proteins resist a detailed structural analysis. Their tendency to form amyloid fibrils prevents the growth of crystals for X-ray studies, and solution NMR spectroscopy for structure determination can so far only be applied for proteins with a molecular weight of up to 40 kDa. However, the fibrils are much larger and, in addition, are insoluble. Solid-state NMR currently represents the only technique for the analysis of PrP^{Sc} in amyloid fibrils at atomic resolution, but this technique still requires tremendous progress with regard to its application to biological macromolecules.

A scientific breakthrough in the investigation of prion diseases is expected from the production and structural characterization of soluble aggregates of the prion protein. According to the common models of prion replication such oligomeric PrP aggregates are of importance for the refolding of the cellular into the infectious scrapie form, and there is some evidence that factor X might participate in this process (Prusiner, 1998). Soluble complexes of PrP^{Sc} as well as PrP^{C}/PrP^{Sc} aggregates are attractive targets for any biochemical or spectroscopic technique in solution. Thus, the development of a protocol for the conformational transmission of recombinant PrP^{C} into PrP^{Sc} would have a multitude of potential applications.

Earlier conversion studies performed with recombinant PrP have shown that no regular protein fibrils are obtained: At acidic pH and in the presence of high concentrations of urea, mPrP(121-231) converts into a soluble β-sheet-rich isoform (Hornemann, S. and Glockshuber, R. (1998) A scrapie-like unfolding intermediate of the prion protein domain PrP(121-231) induced by acidic pH. Proc Natl Acad Sci USA, 95, 6010-6014), whereas hPrP(90-231) in the presence of guanidine hydrochloride converts into a β-sheet-rich isoform that forms fibrillar aggregates (Swietnicki, W., Morillas, M., Chen, S. G., Gambetti, P. and Surewicz, W. K. (2000) Aggregation and fibrillization of the recombinant human prion protein huPrP90-231. Biochemistry, 39, 424-431). However, the ultra structure of these aggregates appears to be not well defined, and it has not been reported whether they show biophysical properties typical for amyloid. Irregular, fibril-like aggregates have also been obtained for hPrP(91-231) under reducing conditions in the absence of detergent (Jackson, G. S., Hosszu, L. L., Power, A., Hill, A. F., Kenney, J., Saibil, H., Craven, C. J., Waltho, J. P., Clarke, A. R. and Collinge, J. (1999) Reversible conversion of monomeric human prion protein between native and fibrilogenic conformations. Science, 283, 1935-1937).

Several different neurodegenerative diseases such as Alzheimer's, Parkinson's and Creutzfeldt-Jacob disease involve the formation of specific proteins or peptides possessing a high content of β-sheet secondary structure, which confers a high tendency for protein/peptide aggregation and formation of very insoluble intra- or extracellular deposits, called amyloid. There is increasing evidence published by leading groups in the field that it is oligomeric versions of such “beta-proteins”, and not necessarily the large aggregates typical of amyloid, that are responsible for triggering pathogenesis of neurodegenerative diseases.

### OBJECT AND SUMMARY OF THE INVENTION

It is therefore one object of the present invention to provide a protocol for producing pathogenic/infectious proteins from recombinant and/or native proteins. This object is attained by the features of claim 1. Particular embodiments of the present invention comprise corresponding methods for proteins or aggregates that are involved in neurodegenerative diseases of the group comprising Transmissible Spongiform Encephalopathy (TSE), Alzheimers disease, Multiple Sclerosis and Parkinsons disease as well as the proteins or protein aggregates produced.

A further object of the present invention is to provide a use of the proteins obtained by these methods including studying the various aspects of the PrP^{C} to PrP^{Sc} conversion under controlled conditions; screening for ligands for the development of a) potential therapeutics against TSE, or b) new diagnostic TSE-tests; development of antibodies specifically binding to (PrP^{Sc}); and determination of the three-dimensional structure of PrP^{Sc} using NMR spectroscopy or X-ray as a basis for the design of ligands.

Still another object of the present invention is to provide a use of the methods according to this invention for the development of potential therapeutics against TSE such as Creutzfeldt-Jakob disease (CJD) in human; the development of antibodies specifically binding to (PrP^{Sc}); for the industrial production of recombinant (PrP^{Sc}); and for the determination of the three-dimensional structure of PrP^{Sc} using NMR spectroscopy or X-ray as a basis for the design of ligands.

Advantageous embodiments and additional characteristics in accordance with the invention ensue from the dependent claims.

This invention includes an in vitro protocol for the generation of a soluble, oligomeric β-sheet-rich conformational variant of recombinant PrP, PrP^{β}, that aggregates into amyloid fibrils, PrP^{βf}, resembling pathogenic PrP^{Sc} in scrapie associated fibrils and prion rods. The conformational transition from PrP to PrP^{βf} occurs at pH 5.0 in bicellar solutions containing equimolar mixtures of dihexanoyl-phosphocholine and dimyristoyl-phospholipids, and a small percentage of negatively charged dimyristoyl-phosphoserine. The protocol was applicable to all species of PrP tested, including human, bovine, elk, pig, dog and murine PrP. Using the N-terminally truncated human PrP fragments hPrP(90-230), hPrP(96-230), hPrP(105-230) and hPrP(121-230) we show that the flexible peptide segment 105-120 is essential for generation of PrP^{β}. Dimerization of PrP represents the rate-limiting step of conversion, which is dependent on the amino acid sequence. The free enthalpy of dimerization is about 130 kJ/mol for human and bovine PrP, and between 260 and 320 kJ/mol for the other species investigated. Hence, the presented in vitro conversion assay allows studying various aspects of transmissible spongiform encephalopathies on a molecular level.

### BRIEF DESCRIPTION OF THE FIGURES

The following figures are intended to document prior art as well as the invention. Preferred embodiments of the method in accordance with the invention will also be explained by means of the figures, without this being intended to limit the scope of the invention.

FIG. 1 Two general models proposed for the molecular mechanism by which PrP^{Sc} promotes the conversion of the cellular isoform (Zahn, R. (1999):

FIG. 1A The “nucleated polymerization” or “seeding” model;

FIG. 1B The “template-assisted” or “heterodimer” model;

FIG. 2 Conformational transition of recombinant mPrP(23-231) into PrP^{β} in bicellar solution as revealed by UV CD:

FIG. 2A PrP refolded into a β-sheet rich form PrP^{β};

FIG. 2B conformational change as observed when 5% dimyristoyl-phosphoglycerol (DMPG) was used instead of DMPS;

FIG. 2C Heating the protein in neutral bicelles, i.e. in the absence of DMPS or DMPG did not induce a change in secondary structure;

FIG. 2D Heating the protein in neutral bicelles, i.e. in lipid-free buffer did not induce a change in secondary structure;

FIG. 3 Dependence of human recombinant PrP to PrP^{β} conversion on the length of the N-terminal “tail”:

FIG. 4A Conversion kinetics of murine PrP measured in conversion buffer as the change in molar ellipticity at 226 nm;

FIG. 4B Doubly logarithmic plot of the initial conversion rates as determined at different temperatures versus the PrP concentration;

FIG. 5 Temperature dependence of PrP to PrP^{β} conversion:

FIG. 5A Transition kinetics of murine PrP;

FIG. 5B Eyring plot of mouse, human, bovine and elk PrP, plotted on a logarithmic scale versus the inverse absolute temperature;

FIG. 6 Sodium dodecylphosphate electrophoresis of recombinant mouse PrP(23-230) after proteinase K digestion:

FIG. 6A PrP^{βf}-aggregates;

FIG. 6B Unconverted PrP;

FIG. 7 Mechanistic model for PrP to PrP^{β} conversion;

FIG. 8 Sequence alignment of mammalian PrP sequences as obtained by the CLUSTAL W algorithm.

### DETAILED DESCRIPTION OF THE INVENTION

The interactions of recombinant PrP expressed in E. coli with lipids have been studied previously. In the presence of high amounts of negatively charged lipids, an alteration of protein secondary structure towards more α-helix (Morillas, M., Swietnicki, W., Gambetti, P. and Surewicz, W. K. (1999) Membrane environment alters the conformational structure of the recombinant human prion protein. J Biol Chem, 274, 36859-36865) or β-sheet structure (Sanghera, N. and Pinheiro, T. J. (2002) Binding of prion protein to lipid membranes and implications for prion conversion. J Mol Biol, 315, 1241-1256) was observed, although no aggregation of PrP into pathogenic amyloid fibrils has been reported in these studies. In an attempt to generate or stabilize amyloidogenic aggregates and β-sheet-rich intermediates of PrP we have studied recombinant protein in bicellar solutions. Bicelles are disc-shaped lipid particles consisting of mixtures of dimyristoyl-phosphocholine (DMPC), dimyristoyl-phosphserine (DMPS) and dihexanoyl-phosphocholine (DHPC). The long chain phospholipids of bicelles form a liquid crystalline bilayered section that is surrounded by a rim of short-chain phospholipids, protecting the long acyl chains from contact with water (Vold, R. R. and i, R. S. (1996) Magnetically oriented phospholipid bilayered micelles for structural studies of polypeptides. Does the ideal bicelle exist? Journal of Magnetic Resonance Series B, 113, 267-271). In the active reconstitution of transmembrane proteins bicelles have been shown to be superior to other compounds (Dencher, N. A. (1989) Gentle and fast transmembrane reconstitution of membrane proteins. Methods Enzymol, 171, 265-274). Moreover, bicelles share some structural features with lipid rafts in that they form disc-shaped lipid bilayers.

Here, we show that bicellar solutions are particularly suitable for the generation of a conformational transition in recombinant PrP into a soluble, oligomeric β-sheet intermediate (PrP^{β}) that can further be converted into amyloid fibrils (PrP^{βf}). These recombinant PrP aggregates essentially show all physico-chemical properties that are documented for PrP^{Sc}. The generation of PrP^{β} starting from recombinant PrP might open an alternative way for studying and exploiting the various aspects of the PrP^{C} to PrP^{Sc} conversion under controlled in vitro conditions.

Furthermore, the invention includes the following applications:

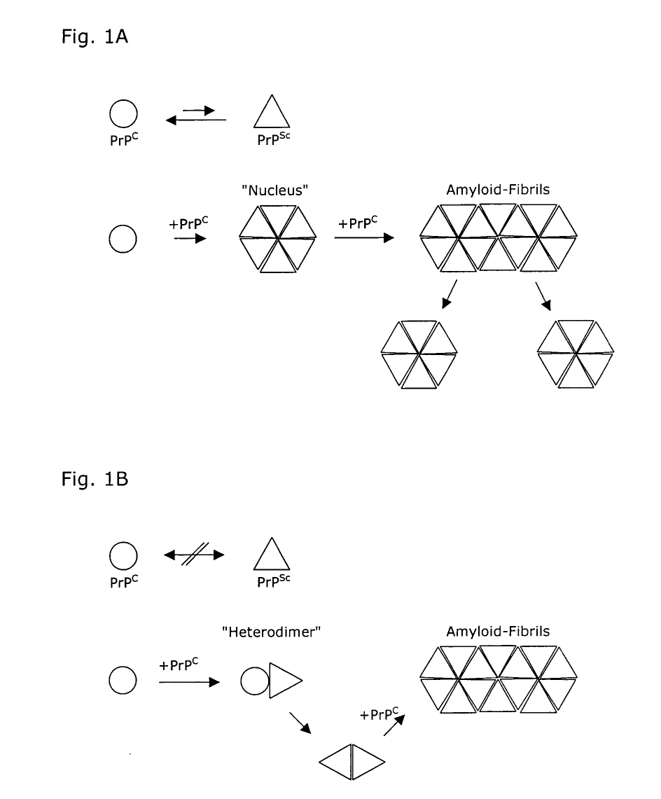

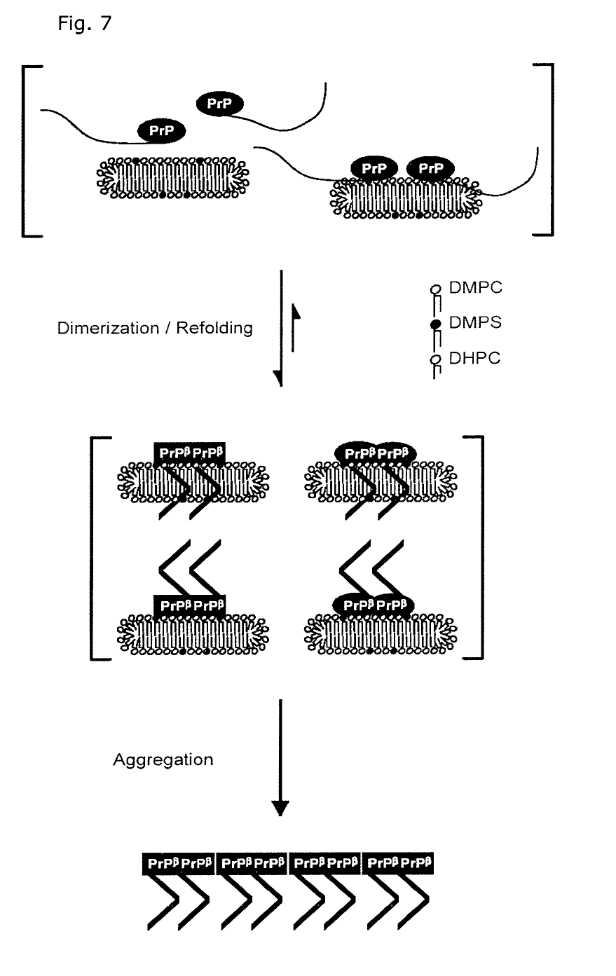

1. The in vitro and in vivo screening for “conversion inhibitors” for the development of potential therapeutics against TSE such as Creutzfeldt-Jakob disease (CJD) in human, where conversion inhibitors include small molecules or biological macromolecules (such as proteins or nucleic acid) that bind to PrP^{C} and thus prevent a conformational transition into PrP^{β} (see FIG. 7) and PrP^{Sc} oligomers (see FIG. 1A) or PrP^{Sc}/PrP^{C} heterodimers (see FIG. 1B). Conversion inhibitors further include small molecules or biological macromolecules that bind to PrP^{β} and PrP^{Sc} oligomers or PrP^{Sc}/PrP^{C} heterodimers, and thus prevent the formation of PrP^{βf} and PrP^{Sc} amyloid fibrils (see FIGS. 1 and 7), conversion inhibitors also include small molecules or biological macromolecules that bind to PrP^{Sc} oligomers, PrP^{β}, and PrP^{βf} and lead to their dissociation into benign isoforms of PrP^{C} oligomers or PrP^{C} monomers. In vitro screening methods include the protocol as summarized in “Object and Summary of the Invention” using CD spectroscopy, electron microscopy, light microscopy and proteinase K resistance assay, but also other spectroscopic techniques such as NMR spectroscopy, dynamic light scattering and fluorescence correlation spectroscopy as well as biochemical techniques such as BIAcore. In vivo screening methods include studies with laboratory animals and cell-culture experiments.

2. The in vitro screening for PrP^{Sc}-specific ligands for the development new diagnostic TSE-tests, where an ideal screening template is represented by PrP^{β} (see FIG. 7). PrP^{Sc}-specific ligands include small molecules or biological macromolecules that bind to PrP^{β} and/or PrP^{βf} (see FIG. 7) and PrP^{Sc} oligomers (see FIG. 1A), PrP^{Sc}/PrP^{C} heterodimers (see FIG. 1B) or PrP^{Sc} amyloid fibrils (see FIG. 1), where the affinity for binding is relatively high when compared to the binding of PrP^{C}. In vitro screening methods include the protocol as summarized in “Object and Summary of the Invention” using electron microscopy, light microscopy and proteinase K resistance assay, but also include other spectroscopic techniques such as dynamic light scattering and fluorescence correlation spectroscopy as well as biochemical techniques.

3. Development of antibodies specifically binding to PrP^{Sc}, where an ideal antigen is represented by PrP^{β} and/or PrP^{βf} (see FIG. 7). Antibodies specifically binding to PrP^{Sc} may be generated by in vitro engineering methods or after active immunization of humans and animals with PrP^{β} or PrP^{βf}. Such antibodies may be applied for passive immunisation of humans and/or animals.”

4. Industrial production of “recombinant PrP^{Sc}” as a “PrP^{Sc} standard” for TSE-tests, where recombinant PrP^{Sc} is represented by PrP^{β} and/or PrP^{βf} (see FIG. 7). A “PrP^{Sc} standard” includes a recombinant PrP standard for measurements on proteinase K resistance and aggregation behaviour using spectroscopic techniques such as dynamic light scattering and fluorescence correlation spectroscopy. TSE-tests may be applied to human and various animals such as cattle, sheep, elk, deer, cat, pig, and horse.

5. Production of “recombinant PrP^{Sc}” for inoculation studies with laboratory animals or cell-culture experiments, where recombinant PrP^{Sc} is represented by PrP^{β} and/or PrP^{βf} (see FIG. 7).

6. Determination of the three-dimensional structure of PrP^{Sc} using NMR spectroscopy, X-ray crystallography or electron microscopy as a basis for the design of ligands and lead compounds. An ideal substrate for NMR in solution and X-ray crystallography is represented by PrP^{β}, and an ideal substrate for solid-state NMR and electron microscopy is represented by PrP^{βf} (see FIG. 7).

7. The invention and its applications may be applied to other proteins involved in neurodegenerative diseases (e.g. Alzheimers, Parkinsons disease, Multiple sclerosis) or generally to proteins causing disease after a conformational transition (conformational diseases such as Primary systematic amyloidosis, Type II diabetes, Atrial amyloidosis).

The invention further includes generation and/or application of wild type proteins according to the points 1-7 or variants thereof. Such variants comprise protein fragments, mutant proteins, fusion proteins, synthetically derived proteins and peptides, and protein-ligand complexes.

### Experimental Results

1. Conversion of Recombinant Murine PrP into PrP^{β}

In conversion buffer containing 25 mM dihexanoyl-phosphocholine (DHPC), 23.75 mM dimyristoyl-phosphocholine (DMPC) and 1.25 mM dimyristoyl-phosphoserine (DMPS), mPrP(23-231) undergoes a conformational transition from a predominantly α-helical into a soluble, β-sheet-rich isoform, termed PrP^{β}.

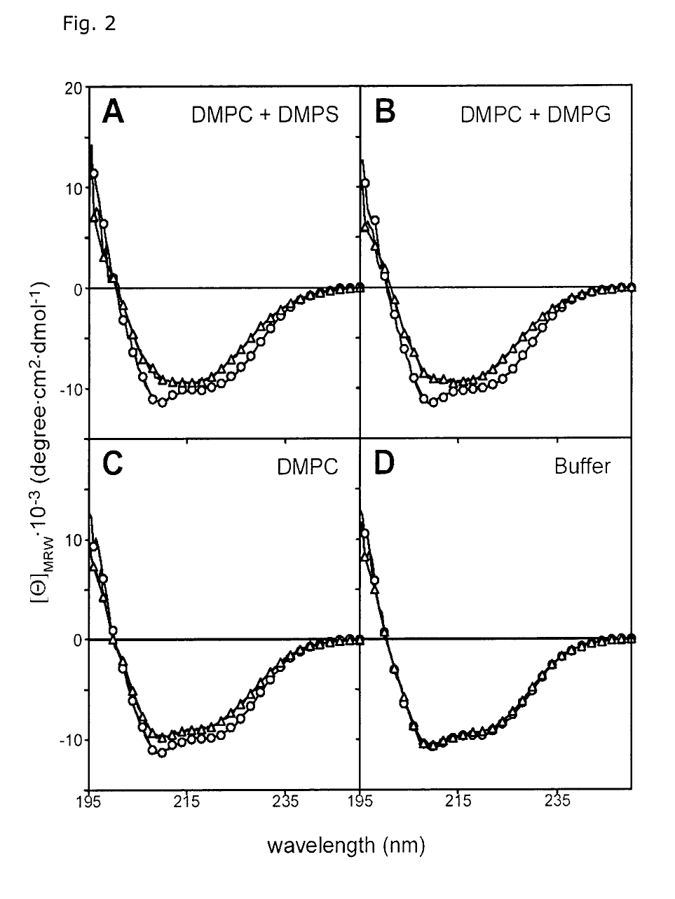

FIG. 2 shows the conformational transition of mPrP(23-231) into PrP^{β} in bicellar solution. The far-UV circular dichroism (CD) spectra were recorded in conversion buffer containing 25 mM long-chain (DMPX; comprising DMPC, DMPG, and/or DMPS) and 25 mM short-chain DHPC phospholipids. First, a spectrum was accumulated at 37° C. (circles), and subsequently the sample was heated to 65° C. for 15 minutes. After equilibration at 37° C., a second CD spectrum was recorded (triangles). FIG. 2A shows that in the presence of 5% DMPS and 95% DMPC, murine PrP refolded into a β-sheet rich form, PrP^{β}, with a characteristic minimum at 215 nm in the CD spectrum. FIG. 2B shows that a similar conformational change was observed when 5% dimyristoyl-phosphoglycerol (DMPG) was used instead of DMPS. FIG. 2C shows that heating the protein in neutral bicelles, i.e. in the absence of DMPS or DMPG did not induce a change in secondary structure. FIG. 2D shows that heating the protein in lipid-free buffer did again not induce a change in secondary structure.

FIG. 2A further shows that at 37° C. the CD spectrum of mPrP(23-231) is characteristic for α-helical secondary structure with a minimum at 208 nm and a shoulder at 217 nm, as has been observed for mPrP(23-231) in the absence of lipids (Hornemann, S., Korth, C., Oesch, B., Riek, R., Wider, G., Wüthrich, K. and Glockshuber, R. (1997) Recombinant full-length murine prion protein, mPrP(23-231): purification and spectroscopic characterization. Febs Letters, 413, 277-281). Heating the protein to 65° C. for 15 minutes leads to the formation of PrP^{β}, which shows a single minimum at 215 nm in the CD spectrum, indicating a relative increase in β-sheet secondary structure. After cooling the sample back to 37° C., only marginal spectroscopic changes were observed. There was no visible aggregation and centrifugation at 20,000 g for 30 minutes did not lead to sedimentation. Moreover, incubation at room temperature for up to 100 days did not significantly alter the CD spectrum. FIG. 2B further shows that substitution of DMPS in bicelles against negatively charged DMPG lead to similar results as compared to FIG. 2A. FIGS. 2C,D further show that heating of mPrP(23-231) in neutral bicelles or lipid-free buffer did not result in the formation of PrP^{β}.

Increasing the relative amount of DMPS to 10% or more appeared to increase the content of α-helix secondary structure in unconverted PrP (data not shown), suggesting that also this form may directly interact with the negatively charged bicelles. However, a fast precipitation upon heating precluded a quantitative analysis of CD spectra. The formation of PrP^{β}, therefore, appears to be an irreversible lipid associated process, which depends on the distribution of negative charges on the lipid bilayer.

2. Conversion of N-Terminally Truncated Human PrP Fragments

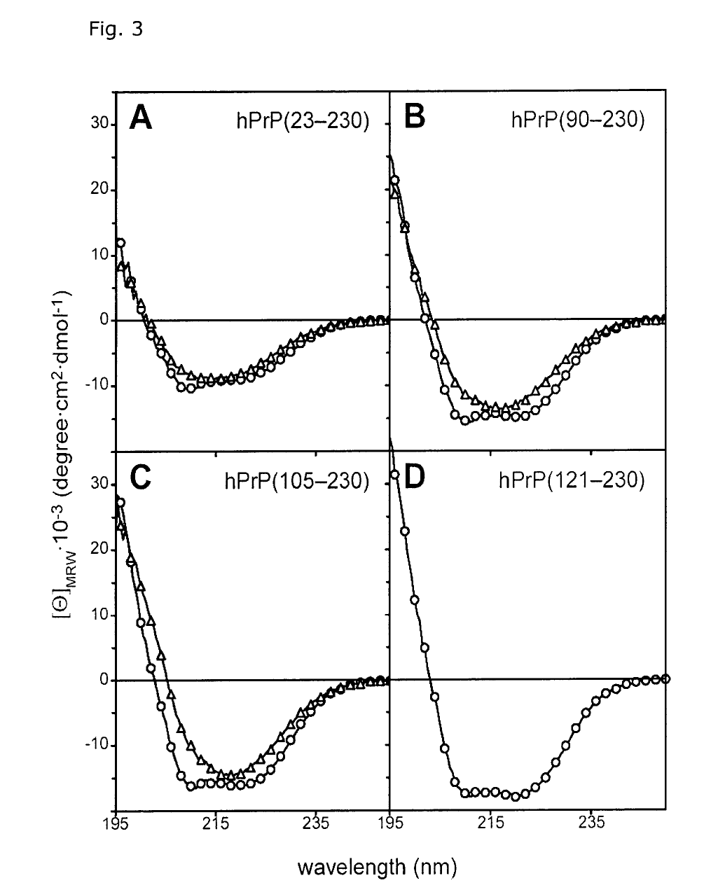

FIG. 3 shows the dependence of human PrP to PrP^{β} conversion on the length of the N-terminal “tail”. CD spectra were recorded as described for FIG. 2: circles, before heating; triangles, after heating. The recombinant PrP constructs are indicated.

In an attempt to narrow down the peptide segment required for the formation of PrP^{β}, we analyzed the spectroscopic properties of intact human PrP and various N-terminally truncated fragments thereof. Upon heating in conversion buffer, hPrP(23-230), hPrP(90-230), and hPrP(105-230) showed a similar transition from mainly α-helical to a β-sheet-rich protein (FIGS. 3A-C). For none of these proteins aggregation was observed upon heating. However, for the fragment hPrP(121-230) heating in conversion buffer immediately led to precipitation so that no meaningful CD spectrum could be recorded (FIG. 3D). The same was observed for mPrP(121-231). Thus, the presence of the peptide segment 105-120 in mammalian PrP appears to be essential for the conformational transition of recombinant PrP into PrP^{β}. Notably, this mostly conserved sequence element among all currently known prion proteins (Wopfner et al., 1999) contains the AGAAAAGA motif, which has been shown to be indispensable for PrP^{C} to PrP^{Sc} conversion in vivo (Holscher, C., Delius, H. and Burkle, A. (1998) Overexpression of nonconvertible PrP^{C} delta114-121 in scrapie-infected mouse neuroblastoma cells leads to trans-dominant inhibition of wild-type PrP(Sc) accumulation. J Virol, 72, 1153-1159).

3. Kinetic Mechanism of Conversion

To get a mechanistic insight into the formation of PrP^{β}, we measured conversion kinetics after rapid heating of the protein solutions at a constant wavelength of 226 nm (see Materials and methods). All kinetic measurements were performed in the presence of 100 mM sodium fluoride to mimic a physiological environment.

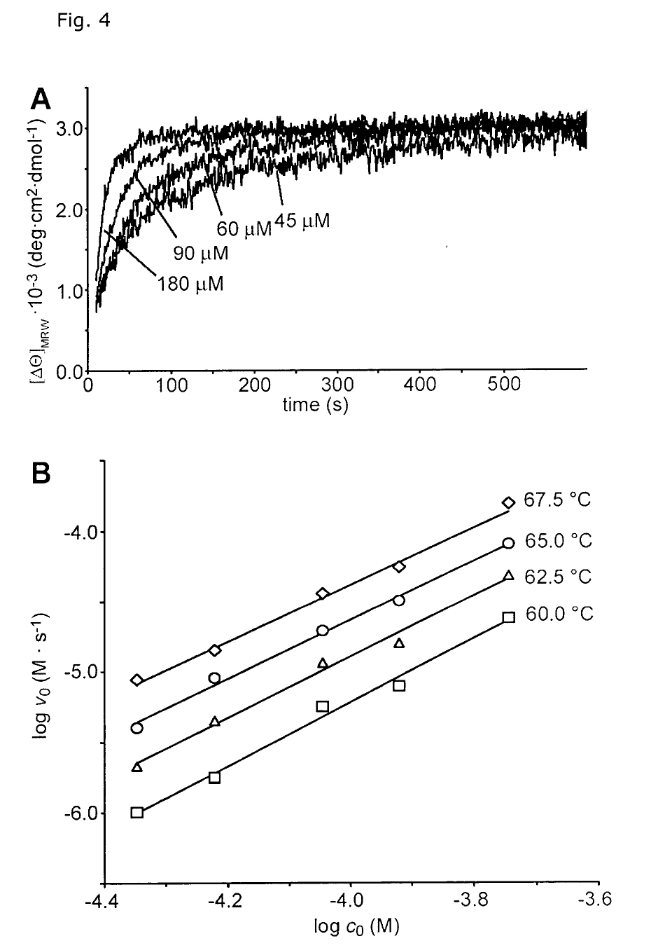

FIG. 4A shows conversion kinetics of murine PrP measured in conversion buffer as the change in molar ellipticity at 226 nm. Varying protein concentrations are indicated next to the corresponding curves. FIG. 4B shows a doubly logarithmic plot of the initial conversion rates as determined at different temperatures versus the PrP concentration (45 to 180 μM).

FIG. 4A further shows a typical data series of conversion kinetics as obtained at different murine PrP concentrations. The reaction becomes significantly faster with increasing protein concentration, suggesting that the conformational change associated with the formation of PrP^{β} occurs in a cooperative manner involving oligomerization of PrP molecules. There was no increase in the observed rate constant, when the conversion was performed in the presence of catalytic concentrations of preformed PrP^{β}. In FIG. 4B, the logarithms of the initial reaction rates is plotted against the logarithm of the protein concentration. Independently of the temperature, the slope of these curves is n=2.1±0.2. Thus, a dimerization seems to be the rate-limiting step for the transition of monomeric PrP to oligomeric PrP^{β}.

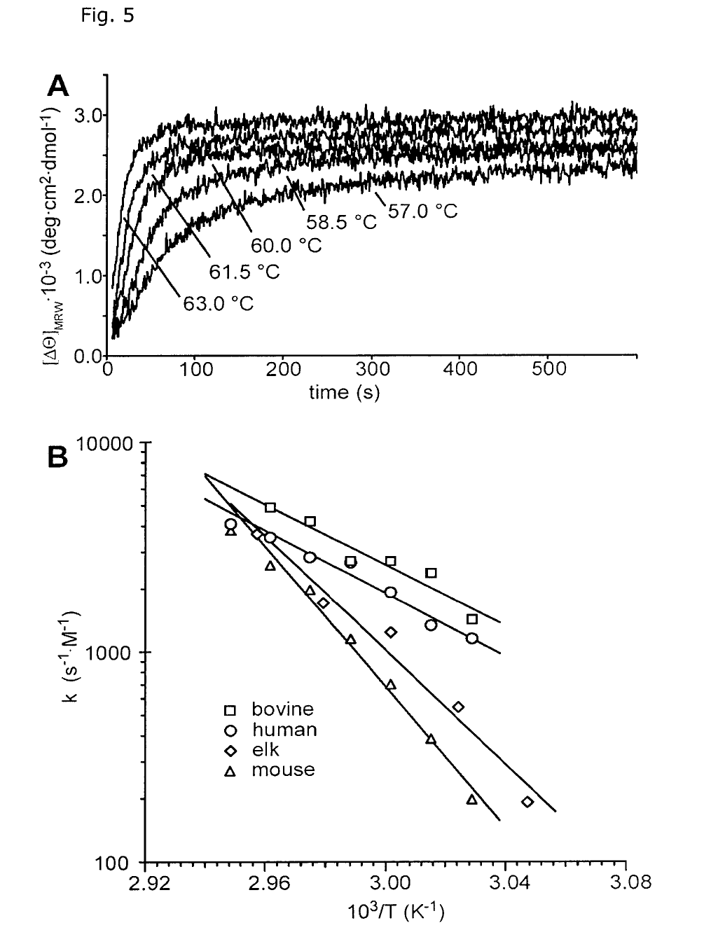

FIG. 5 shows the temperature dependence of PrP to PrP^{β} conversion. According to FIG. 5A transition kinetics of murine PrP were measured at a constant protein concentration of 100 μM at various temperatures between 57° C. and 65° C. FIG. 5B shows an Eyring plot of mouse, human, bovine and elk PrP, where the rate constants for conversion, k, were plotted on a logarithmic scale versus the inverse absolute temperature.

Inspection of FIG. 5A shows that the reaction rate increases with temperature so that the activation enthalpy associated with the rate-limiting step for conversion can be determined according to the Eyring equation. The logarithmic plot of the reaction rate constant, k, versus the inverse absolute temperature, and the fit of the experimental data are shown in FIG. 5B. The calculated activation parameters for various fragments and species of PrP are summarized in Table 1.

TABLE 1
Kinetic parameters of PrP to PrP^{β} conversion experiments.

| | | | ΔH^{≠2} | ΔS^{≠2} | ΔG^{≠3} | |
|---|---|---|---|---|---|---|
| | | | (kJ/ | (J/ | (kJ/ | k⁴ |
| Species | Fragment | Conversion¹ | mol) | K · mol) | mol) | (s⁻¹ · M⁻¹) |
| human | 23-230 | yes | 130 | 200 | 66 | 60 |
| | 90-230 | yes | 140 | 230 | 67 | 40 |
| | 96-230 | yes | | | | |
| | 100-230 | yes | 140 | 230 | 67 | 40 |
| | 105-230 | yes | | | | |
| | 121-230 | no | | | | |
| bovine | 25-242 | yes | 140 | 230 | 65 | 60 |
| elk | 25-234 | yes | 260 | 580 | 76 | 0.9 |
| pig | 25-235 | yes | 260 | 600 | 77 | 0.7 |
| dog | 25-233 | yes | 310 | 730 | 80 | 0.2 |
| mouse | 23-231 | yes | 320 | 760 | 81 | 0.1 |
| | 23-231⁵ | yes | 250 | 570 | 75 | 2 |
| | 121-231 | no | | | | |

¹PrP to PrP^{β} conversion as evidenced by characteristic β-sheet CD spectra and the absence of precipitation after heating in conversion buffer (see Materials and methods).
²Values were obtained by fitting equation 5 to experimentally determined reaction rates k at various temperatures.
^{3,4}Calculated at 37° C. using equations 6 and 5, respectively.
⁵The conversion was performed in the presence of 2 M Urea.

For murine and dog PrP, activation enthalpies of about 300 kJ·mol⁻¹ were obtained, which is about twice as high, as the corresponding enthalpies of intact human and bovine PrP. This finding correlates with the notion that the NMR structures of human and bovine PrP are closely similar, while they both differ significantly from the structure of murine PrP (Lopez Garcia et al., 2000).

4. Generation of Recombinant PrP Fibrils: PrP^{βf}

The detergent DHPC constitutes a major component of the bicelles in the conversion buffer. In mixtures with long-chain phospholipids, the critical micelle concentration (cmc) of DHPC is approximately 5 mM (Ottiger, M. and Bax, A. (1998) Characterization of magnetically oriented phospholipid micelles for measurement of dipolar couplings in macromolecules. J Biomol NMR, 12, 361-372), and below this concentration the long chain phospholipids form vesicles, both at moderately acidic or at neutral pH (Ottiger, M. and Bax, A. (1999) Bicelle-based liquid crystals for NMR-measurement of dipolar couplings at acidic and basic pH values. J Biomol NMR, 13, 187-191). In our conversion assay, dilution of PrP^{β}-bicellar solutions significantly below the cmc of DHPC immediately resulted in precipitation of PrP^{β} into PrP^{βf}. Treatment of these aggregates with non-denaturing detergents such as octylglucoside led to the formation of regular fibrils, PrP^{βf}, which could be observed in the electron microscope. Similar fibrils were observed when PrP^{β} was directly treated with detergent without previous dilution of the lipids.

Electron microscopy has been carried out on detergent treated PrP amyloid fibrils: 25 μM mouse PrP^{βf} was sedimented at 20,000 g and resuspended in 50 mM Tris-HCl, 150 mM NaCl, 320 mM sucrose and 0.5% (w/v) octylglucoside. The amyloid fibrils produced have a tendency to form large bundles. However, also single fibrils consisting of two or four helically wound proto-filaments with a diameter of 10.5±0.6 nm and 25.8±0.6 nm, respectively were also observed (data not shown). These proto-filaments contain a beaded substructure with a diameter of 4 to 4.5 nm.

Similar substructures have been described for scrapie associated fibrils, and it has been speculated that they might represent subunits of the fibrils (Merz, P. A., Somerville, R. A., Wisniewski, H. M. and Iqbal, K. (1981) Abnormal fibrils from scrapie-infected brain. Acta Neuropathol (Berl), 54, 63-74). Assuming a spherical shape, a single bead of PrP^{βf} contains a volume of 34-48 nm³ corresponding to 1.7-2.5 times the volume of hPrP(90-230). This points towards the observation that the rate-limiting step in the formation of PrP^{β} is dimerization. Thus, PrP^{βf} might represent polymeric aggregates of PrP dimers, and possibly also scrapie associated fibrils may consist of similar building blocks.

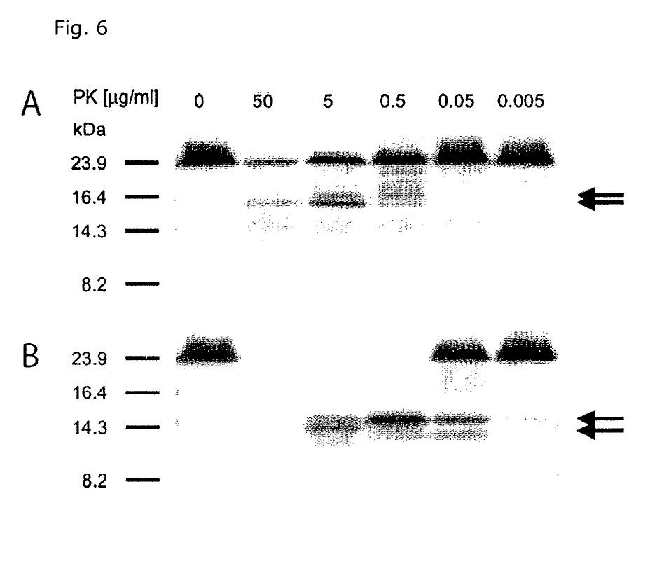

We found that PrP^{βf} binds congo-red and shows green-gold birefringence in cross-polarized light (data not shown), and that it contains a partially proteinase K resistant core corresponding to bona fide PrP^{Sc} (see FIG. 6).

FIG. 6 shows the result of sodium dodecylphosphate electrophoresis of recombinant mouse PrP(23-230) after proteinase K digestion. FIG. 6A shows PrP^{βf}-aggregates. Arrows indicate proteolytic fragments between 16.0 and 16.4 kDa, corresponding to PrP residues 105-230 and 99-230, respectively. FIG. 6B shows unconverted PrP. Arrows indicate major proteolytic fragments between 13.5 and 14.7 kDa.

### Discussion of Results

1. The Mechanism of PrP Conversion

A possible mechanism for the formation of PrP^{β} in bicellar solution is shown in FIG. 7.

FIG. 7 shows a mechanistic model for PrP to PrP^{β} conversion. Recombinant PrP is represented by an ellipsoid (residues 121-230) and a random line (residues 90-120). In PrP^{β}, the flexible tail becomes structured as indicated by the geometric line. The structure of the globular domain in PrP^{β} is either preserved, or participates in the α-helix to β-sheet conformational transition (rectangle). The relative dimensions of bicelles composed of lipid molecules and protein molecules are approximately to scale.

The observation that negative charges have to be present on the bilayer surface of bicelles for efficient conversion argues that the first step in the reaction is an electrostatic adsorption of PrP to the bilayer/water interface. This view is supported by previous observations of the partitioning of recombinant prion proteins to negatively charged bilayers (Morillas et al., 1999; Sanghera and Pinheiro, 2002). The PrP concentration dependence of the conversion reaction suggests that all other rates along the reaction pathway must be significantly faster than dimerization of PrP. Moreover, dimerization per se does not lead to an observable change in the CD spectrum, while refolding does. Hence, the conformational transition of PrP to PrP^{β} is coupled to protein dimerization. We estimate that the bicelles used in our assay have a diameter of about 10 nm (Vold and Prosser, 1996), which would provide enough space to accommodate 10 to 20 PrP monomers depending on the orientation of PrP molecules relative to the bicellar membranes. Upon dilution of DHPC beyond the cmc or in the presence of detergents, individual particles would meet, leading to the formation of highly polymeric PrP aggregates, PrP^{βf}. This model of PrP conversion is in agreement with the observations made by Caughey and co-workers in a cell-free conversion reaction (Baron, G. S., Wehrly, K., Dorward, D. W., Chesebro, B. and Caughey, B. (2002) Conversion of raft associated prion protein to the protease-resistant state requires insertion of PrP-res (PrP(Sc)) into contiguous membranes. Embo J, 21,1031-1040), suggesting that the generation of new PrP^{Sc} during TSE infection requires: (i) removal of PrP^{C} from target cells; (ii) an exchange of membranes between cells; or (iii) insertion of incoming PrP^{Sc} into the lipid raft domains of recipient cells.

The possible modes of interaction of PrP with bicelles include the adsorption to the bilayer surface and the formation of transmembrane segments by sideward insertion through the rim of DHPC. The requirement of the hydrophobic peptide segment 112-130 for the conversion to occur argues in favor of the view that this part of PrP inserts into the bilayer, although it is also possible that PrP^{β} is only adsorbed to the lipid surface. If the conformational transition is accompanied by the formation of β-sheet secondary structure within the flexibly disordered tail or the globular domain or both cannot be readily decided from our current data (see FIG. 7). However, the fact that the peptide segment 90-120 becomes proteinase K resistant after conversion indicates that the tail is involved in the conformational transition. Further valuable information is provided by the transition state energetics of PrP^{β} formation collected in Table 1. All transition state entropies have large positive values, indicating that the transition state contains a higher degree of disorder as compared to unconverted PrP. The peptide segment 105-120 is flexibly disordered in unconverted PrP, making it unlikely to contribute positively to ΔS^{≠}. These data suggest that the flexible tail, but also partial unfolding of the globular domain 121-230 features in the conversion process, which would be consistent with the decreased α-helix and increased p-sheet secondary structure observed in FIGS. 2A,B and 3A-C. This model appears plausible, as the peptide segment 110-140 has been demonstrated to traverse lipid bilayers in transmembrane forms of PrP that are presumably involved in pathogenesis and amplification of the TSE agent (Hegde, R. S., Mastrianni, J. A., Scott, M. R., DeFea, K. A., Tremblay, P., Torchia, M., DeArmond, S. J., Prusiner, S. B. and Lingappa, V. R. (1998) A transmembrane form of the prion protein in neurodegenerative disease. Science, 279, 827-834; Hegde, R. S., Tremblay, P., Groth, D., DeArmond, S. J., Prusiner, S. B. and Lingappa, V. R. (1999) Transmissible and genetic prion diseases share a common pathway of neurodegeneration. Nature, 402, 822-826). Because two third of this peptide segment are structured within the PrP^{C} scaffold, such membrane-associated forms most likely contain a structurally altered globular domain.

2. Implications for the Species Barrier of TSE Transmission

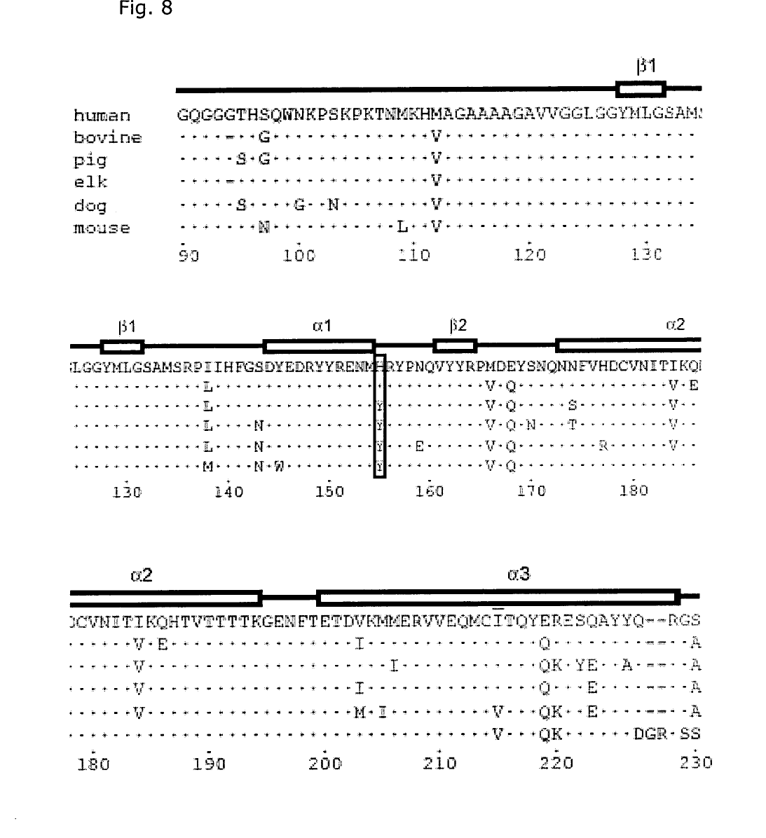

Large differences in the activation enthalpies of the PrP to PrP^{β} conversion are observed between the two groups of mammalian prion proteins, including human and bovine PrP, and elk, pig, dog and mouse PrP, respectively (Table 1). The relatively low activation entropies of intact human and bovine PrP argue that the transition state(s) is less unfolded compared to the other prion proteins. Moreover, from the calculated free energy values of conversion and the corresponding reaction rate constants, estimated at 37° C., it turns out that spontaneous conversion in human and bovine PrP is about 600 times faster than in e.g. mouse PrP. Notably, human and bovine PrP are mostly similar with regard to the amino acid sequence and the three-dimensional structure (Lopez Garcia et al., 2000). As the only difference in the conversion reactions is the amino acid sequence of PrP, the variations in kinetic parameters must be rationalized on the basis of species-specific amino acid variations. Consistent sequence variations between the two aforementioned PrP groups are found only in position 155, where human and bovine PrP contain a histidine as compared to tyrosine in the other prion proteins (FIG. 8).

FIG. 8 shows the sequence alignment of mammalian PrP sequences as obtained by the CLUSTAL W algorithm (version 1.8; (Thompson, J. D., Higgins, D. G. and Gibson, T. J. (1994) Clustal-W—Improving the Sensitivity of Progressive Multiple Sequence Alignment through Sequence Weighting, Position-Specific Gap Penalties and Weight Matrix Choice. Nucleic Acids Research, 22, 4673-4680) ordered with increasing activation enthalpy of conversion (see Table 1) from top to bottom. The identities of individual sequences are indicated on the left. At the top, secondary structure elements of human PrP (Zahn, R., Liu, A., Luhrs, T., Riek, R., von Schroetter, C., Lopez Garcia, F., Billeter, M., Calzolai, L., Wider, G. and Wüthrich, K. (2000) NMR solution structure of the human prion protein. Proc Natl Acad Sci USA, 97, 145-150) are indicated: empty boxes, regular secondary; black line, non-regular secondary structure. The residue numbers according to human PrP are indicated at the bottom.

The protonation of solvent exposed His155 (Zahn, R., Liu, A., Luhrs, T., Riek, R., von Schroetter, C., Lopez Garcia, F., Billeter, M., Calzolai, L., Wider, G. and Wüthrich, K. (2000) NMR solution structure of the human prion protein. Proc Natl Acad Sci USA, 97, 145-150) appears to substantially increase the population of transition competent protein conformations that are able to convert into PrP^{β}. The impact of His155 on conversion of recombinant PrP is intriguing as cell-free conversion experiments with chimeric mouse/hamster PrP have shown that the PrP^{Sc} epitope of hamster PrP^{C} includes Met139, Asn155 and Asn170 (Kocisko, D. A., Priola, S. A., Raymond, G. J., Chesebro, B., Lansbury, P. T., Jr. and Caughey, B. (1995) Species specificity in the cell-free conversion of prion protein to protease-resistant forms: a model for the scrapie species barrier. Proc Natl Acad Sci USA, 92, 3923-3927). Thus, the conformational transition and dimerization of PrP into PrP^{β} observed in our conversion assay appears to reflect the conversion of native PrP^{C} into PrP^{Sc}. If so, one comes to the conclusion that the species barrier for transmission of TSE between human and cattle presumably is less stringent than for the other species investigated.

3. Implications for Familial CJD Forms

Single amino acid substitutions in the globular domain of human PrP have been shown to segregate with familial CJDs (for review (Prusiner, 1998)). However, mechanistic details about this process are not known. Unlike in most folding experiments, where the transition between unfolded and folded states of proteins is studied, the transition of PrP to PrP^{β} occurs between two folded conformations. Thus, familial amino acid substitutions may affect the three-dimensional structure of the native state, transition state, or converted state of PrP. The impact of familial CJD variations on thermodynamic stability has previously been studied with recombinant murine PrP (Liemann, S. and Glockshuber, R. (1999) Influence of amino acid substitutions related to inherited human prion diseases on the thermodynamic stability of the cellular prion protein. Biochemistry, 38, 3258-3267). While five of the amino acid replacements destabilized the native state of PrP(121-231), three other variants had virtually no effect on thermodynamic stability. Moreover, a spontaneous formation of PrP^{Sc}-like aggregates was not observed for the destabilized variants, suggesting that an unfolding of the PrP^{C} conformation alone is not sufficient for the generation of PrP^{Sc}. These results are in agreement with our conversion experiments carried out in the presence of 2M urea (Table 1), showing that the effect of high concentrations of denaturant on transition state parameters is much lower compared to substitution of a single amino acid residue, e.g. of Tyr at position 155 against His.

The presence of additional octapeptide segments in the amino acid sequence of human PrP has been demonstrated to segregate with a heritable risk to develop familiar CJD, and up to nine additional octapeptide repeats have been found in humans (Goldfarb, L. G., Brown, P., McCombie, W. R., Goldgaber, D., Swergold, G. D., Wills, P. R., Cervenakova, L., Baron, H., Gibbs, C. J., Jr. and Gajdusek, D. C. (1991) Transmissible familial Creutzfeldt-Jakob disease associated with five, seven, and eight extra octapeptide coding repeats in the PRNP gene. Proc Natl Acad Sci USA, 88, 10926-10930). Each octapeptide repeat contains a tryptophane, which is an amino acid that preferentially partitions to the lipid/water interface. Thus, this sequence motif might promote conversion by binding to the membrane surface and leading to a local increase of PrP concentration. However, truncation of residues 23-88 comprising the N terminus of mature PrP does not prevent PrP^{Sc} synthesis in transgenic mice (Fischer, M., Rulicke, T., Raeber, A., Sailer, A., Moser, M., Oesch, B., Brandner, S., Aguzzi, A. and Weissmann, C. (1996) Prion protein (PrP) with amino-proximal deletions restoring susceptibility of PrP knockout mice to scrapie. Embo J, 15, 1255-1264) and in ScN₂a cells (Rogers, M., Yehiely, F., Scott, M. and Prusiner, S. B. (1993) Conversion of truncated and elongated prion proteins into the scrapie isoform in cultured cells. Proc Natl Acad Sci USA, 90, 3182-3186), indicating that the octapeptide region is not required for prion propagation, although incubation times in transgenic mice are longer than in wild-type mice (Flechsig, E., Shmerling, D., Hegyi, I., Raeber, A. J., Fischer, M., Cozzio, A., von Mering, C., Aguzzi, A. and Weissmann, C. (2000) Prion protein devoid of the octapeptide repeat region restores susceptibility to scrapie in PrP knockout mice. Neuron, 27, 399-408). These finding are reflected by our observation (Table 1) that at 37° C. the reaction rate constant of intact human PrP is only slightly higher than the rate constants of N-terminally truncated human prion proteins that lack the octapeptide repeats.

### Materials and Methods

1. Buffers and Solutions

CB=conversion buffer

(25 mM DHPC, 23.75 mM DMPC, 1.25 mM DMPS, 50 mM sodium acetate pH 5.0, 100 mM sodium fluoride);

NaAc=sodium acetate buffer

(50 mM sodium acetate pH 5.0);

TNO=Tris-HCl/octylglucoside buffer (25 mM Tris-HCl pH 7.5, 150 mM NaAc, 10/% (w/v) Octylglucoside);

TNSucO=TNO containing 0.32 M sucrose.

2. Purification of Prion Protein

Recombinant prion proteins were expressed and purified as described previously (Zahn, R., Liu, A., Luhrs, T., Riek, R., von Schroetter, C., Lopez Garcia, F., Billeter, M., Calzolai, L., Wider, G. and Wüthrich, K. (2000) NMR solution structure of the human prion protein. Proc Natl Acad Sci USA, 97, 145-150; Zahn, R., von Schroetter, C. and Wüthrich, K. (1997) Human prion proteins expressed in Escherichia coli and purified by high-affinity column refolding. FEBS Lett, 417, 400-404), and their identities was confirmed by DNA sequencing, N-terminal amino acid sequencing and MALDI-TOF mass-spectrometry.

3. CD Spectroscopy

Measurements were performed using a 0.2 mm quartz cuvette on a Jasco J-815 spectropolarimeter equipped with a PFD-350S temperature control unit. CD spectra were measured with 50 μM PrP in CB containing no sodium fluoride. Typically 10 scans with data intervals of 0.5 nm and a response time of 1 second were accumulated at a speed of 10 nm/min. Kinetic measurements were performed by rapid heating of 45-180 μM PrP in CB, and tracing the change in ellipticity at a wavelength of 226 nm. The data interval and the response time were 1 second, and a bandwidth of 4 nm was used. As a baseline for unconverted PrP, kinetics was acquired at 37° C. The temperature dependence of conversion was measured in a temperature range of 55-65° C. using 100 μM PrP in CB.

4. Data Analysis

Kinetic data were analyzed assuming an oligomerization of the type n·PrP→PrPₙ, where n denotes the number of PrP monomers per cooperative unit. Formally, this reaction is described by the equation

dc/dt=−k·cⁿ [1]

where c, t, and k denote the PrP concentration, the time, and the reaction rate constant, respectively. The general solution of equation 1 is:

c(t)={c₀⁽¹⁻ⁿ⁾−(n−1)·k·t}^{1/(1-n)} [2].

At t=0, the protein concentration is equal to the initial concentration, c₀, and the initial reaction rate, v₀, can be written as

v₀=k·c₀ⁿ [3]

or

log(v₀)=n·log(c₀)+log(k) [4].

Rate constants were obtained by fitting the kinetic data to equation 2 with n=2 and using k as the fitting parameter. The activation barrier associated with a rate-limiting step is described by the Eyring equation:

k(T)=k_{b}T/h·exp(ΔS^{≠}/R)·exp(−H^{≠}/RT) [5],

where k_{b}, h, ΔS^{≠}, and ΔH^{≠} denote the Boltzmann constant, the Planck constant, the activation entropy, and the activation enthalpy, respectively. ΔS^{≠} and ΔH^{≠} were then obtained by fitting eq. 5 to experimental values of k(T). From these values the free energy of activation was calculated as

ΔG^{≠}=ΔH^{≠}−ΔT·ΔS^{≠} [6].

4. Preparation of PrP Amyloid Fibrils

Recombinant murine PrP (50-250 μM) in CB was heated for 15 minutes to 65° C. and allowed to cool to room temperature (RT) for 15 minutes, yielding PrP^{β}. Subsequently, aggregation was induced by addition of nine volumes NaAc, yielding PrP^{βf}. After 60 minutes aggregated material was collected by centrifugation at 20,000 g for 15 minutes.

5. Proteinase K Digestion

Protease resistance of recombinant PrP(23-230) was determined at a protein concentration of 100 μM in the presence of 0 to 50 μg/ml proteinase K at 37° C. in buffer solution containing 50 mM sodium phosphate pH 7.0 and 150 mM sodium chloride. After 60 minutes protein was collected for sodium dodecylphosphate gel electrophoresis.

6. Electron Microscopy

Freshly carbon coated EM grids (400 MESH) were layered on top of one drop of PrP^{βf} suspended either in TNO or TNSucO. After incubation for one minute at RT, excess liquid was carefully removed from the grid using a filter paper, before washing with three drops of distilled water. The amyloid fibril containing EM grid was stained for one minute with one drop of 2% (w/v) uranylacetate, and was analyzed on a Philips H600 electron microscope at 100 kV with magnifications between 10,000× and 30,000×.

What is claimed is:

1. An in vitro method for inducing a conformational transition in recombinant proteins and/or variants thereof, whereas said conformational transition results in an increased content of β-sheet secondary structure, the method comprising the steps of:
a) providing a conversion buffer;
b) adding a solution of lamellar lipid structures that comprise negatively charged lipids to the conversion buffer;
c) adding recombinant protein molecules and/or variants thereof to the conversion buffer;
d) forming a sample mixture from the conversion buffer, the added lipids and protein molecules;
e) establishing a conversion temperature in the sample mixture; and
f) exposing the sample mixture of step d) to the conversion temperature according to step e) for a time sufficient to form conformationally transitioned proteins; wherein
water soluble complexes of lamellar lipidic structures and conformationally transitioned proteins are formed, the conformationally transitioned proteins being oligomeric β-sheet intermediate structures.

2. Method according to claim 1, wherein amyloidogenic aggregates are produced of the water soluble complexes of lamellar lipidic structures and oligomeric β-sheet intermediate structures by actively destroying the lamellar lipid structures.

3. Method according to claim 2, wherein for producing said amyloidogenic aggregates, the lamellar lipid structures of the water soluble complexes are destroyed by:
a) dilution of the solution of the water soluble complexes of lamellar lipidic structures and oligomeric intermediate structures significantly below the critical micelle concentration of the lipids used; or
b) dilution of the solution of the water soluble complexes of lamellar lipidic structures and oligomeric intermediate structures significantly below the critical micelle concentration of the lipids used and treatment of the so produced amyloidogenic aggregates with non-denaturing detergents such as octylglucoside; or
c) directly treating the water soluble complexes of lamellar lipidic structures and oligomeric intermediate structures with detergent, without previous dilution of the lipids.

4. Method according to claim 1, wherein the solution of lamellar lipid structures in step b) is a bicellar lipid solution; in step e) the sample mixture is heated to a conversion temperature which is higher than the temperature for forming the sample mixture, and the water soluble oligomeric β-sheet intermediate structure is an oligomeric β-sheet intermediate (PrP^{β}) which is aggregated into amyloid fibrils (PrP^{βf}).

5. Method according to claim 4, wherein the sample mixture is heated in step e) to a conversion temperature in the range from 37° C. to 65° C.

6. Method according to claim 4, wherein these proteins are involved in:
a) neurodegenerative diseases of the group comprising Transmissible Spongiform Encephalopathy (TSE), Alzheimers disease, Multiple Sclerosis, and Parkinsons disease; and/or other
b) conformational diseases of the group comprising Primary systematic amyloidosis, Type II diabetes, and Atrial amyloidosis.

7. The method of claim 1, wherein the conversion buffer comprises 25 mM long-chain (DMPX) and 25 mM short-chain (DHPC) phospholipids to form a bicellar solution.

8. The method of claim 7, wherein the long-chain phospholipid in the conversion buffer is 23,75 mM (DMPC) and 1.25 mM (DMPS or DMPG), and wherein the short-chain phospholipid is 25 mM (DHPC).

9. The method of claim 1, wherein the pH of the conversion buffer is below the isoelectric point of the recombinant proteins.

10. Oligomeric β-sheet intermediate structures produced by an in vitro method for inducing a conformational transition in recombinant proteins and/or variants thereof, whereas said conformational transition results in an increased content of β-sheet secondary structure, the method comprising the steps of:
a) providing a conversion buffer;
c) adding a solution of lamellar lipid structures that comprise negatively charged lipids to the conversion buffer;
c) adding recombinant protein molecules and/or variants thereof to the conversion buffer;
d) forming a sample mixture from the conversion buffer, the added lipids and protein molecules;
e) establishing a conversion temperature in the sample mixture; and
f) exposing the sample mixture of step d) to the conversion temperature according to step e) for a time sufficient to form conformationally transitioned proteins; wherein
the oligomeric β-sheet intermediate structures are conformationally transitioned proteins that are part of water soluble complexes comprising lamellar lipidic structures.

11. Amyloidogenic aggregates produced from the oligomeric β-sheet intermediate structures of claim 10, wherein the amyloidogenic aggregates are derived of the oligomeric β-sheet intermediate structures by actively destroying the lamellar lipid structures of the water soluble complexes of lamellar lipidic structures and conformationally transitioned proteins.

12. Use of the water soluble complexes of oligomeric β-sheet intermediate structures and lamellar lipidic structures according to claim 10 or of amyloidogenic aggregates according to claim 11, for exploiting the various aspects of the conversion under controlled conditions.

13. Use of the method according to claim 6, for the screening for “conversion inhibitors” for the development of diagnostics and/or prophylactics and/or therapeutics against conformational diseases in humans and/or animals.

14. Use of the water soluble complexes of oligomeric β-sheet intermediate structures and lamellar lipidic structures according to claim 10 or of amyloidogenic aggregates according to claim 11, for the screening for “conversion inhibitors” for the development of diagnostics and/or prophylactics and/or therapeutics against conformational diseases in humans and/or animals.

15. Use of the water soluble complexes of oligomeric β-sheet intermediate structures and lamellar lipidic structures according to claim 10 or of amyloidogenic aggregates according to claim 11, for the screening for ligands for the development of
a) therapeutics against conformational diseases in humans and/or animals; or
b) prophylactics against conformational diseases in humans and/or animals; or
c) diagnostic tests for conformational diseases in humans and/or animals.

16. Use of the method according to claim 6, for the screening for ligands for the development of
a) therapeutics against conformational diseases in humans and/or animals; or
c) prophylactics against conformational diseases in humans and/or animals; or
c) diagnostic tests for conformational diseases in humans and/or animals.

17. Use of the water soluble complexes of oligomeric β-sheet intermediate structures and lamellar lipidic structures according to claim 10 or of amyloidogenic aggregates according to claim 11, for the development of antibodies specifically binding to conformationally transitioned proteins.

18. Use of the method according to claim 6, for the development of antibodies specifically binding to conformationally transitioned proteins.

19. Use of the method according to claim 1, for the industrial production of recombinant conformationally transitioned proteins.

20. Use of the water soluble complexes of oligomeric β-sheet intermediate structures and lamellar lipidic structures according to claim 10 or of amyloidogenic aggregates according to claim 11, for the determination of the three-dimensional structure of conformationally transitioned proteins using NMR spectroscopy, X-ray, or electron microscopy as a basis for the design of ligands.

21. Use of the method according to claim 1, for the determination of the three-dimensional structure of oligomeric β-sheet intermediate structures of the conformationally transitioned proteins and/or amyloidogenic aggregates using NMR spectroscopy, X-ray, or electron microscopy as a basis for the design of ligands.

22. Use of ligands as derived according to claims 15, 16, 20 or 21, for diagnosis and/or prophylactics and/or therapeutic treatment of conformational diseases in humans and/or animals.

23. Use of “conversion inhibitors” as derived according to claims 13 or 14, for diagnosis and/or prophylactics and/or therapeutic treatment of conformational diseases in humans and/or animals.

24. Use of antibodies as derived according to claims 17 or 18, for diagnosis and/or prophylactics and/or therapeutic treatment of conformational diseases in humans and/or animals.

25. Use of the water soluble complexes of oligomeric β-sheet intermediate structures and lamellar lipidic structures according to claim 10 or of amyloidogenic aggregates according to claim 11, for the active immunization of humans or animals against neurodegenerative diseases of the group comprising Transmissible Spongiform Encephalopathy (TSE), Alzheimers disease, Multiple Sclerosis, and Parkinsons disease; and/or other conformational diseases of the group comprising Primary systematic amyloidosis, Type II diabetes, and Atrial amyloidosis.

26. Use of antibodies as derived according to claims 17 or 18 for the manufacture of a medicament for passive immunisation of humans or animals against neurodegenerative diseases of the group comprising Transmissible Spongiform Encephalopathy (TSE), Alzheimers disease, Multiple Sclerosis, and Parkinsons disease; and/or other conformational diseases of the group comprising Primary systematic amyloidosis, Type II diabetes, and Atrial amyloidosis.

27. Composition for diagnosis or therapeutical or prophylactical treatment of human and/or animal conformational diseases, comprising “conversion inhibitors” as obtained according to the method of claim 13 or 14.

28. Composition for diagnosis or therapeutical or prophylactical treatment of human and/or animal conformational diseases, comprising ligands as obtained according to the method of claim 15, 16, 20 or 21.

29. Composition for diagnosis or therapeutical or prophylactical treatment of human and/or animal conformational diseases, comprising antibodies specifically binding to conformationally transitioned proteins as obtained according to the method of claim 17 or 18.
